(19) United States
(12) Patent Application Publication
(10) Pub. No.: US 2018/0318501 A1
(43) Pub. Date: Nov. 8, 2018

(54) METHOD AND APPARATUS FOR PERFORMING A PERIPHERAL NERVE BLOCK

(71) Applicant: Milestone Scientific, Inc., Livingston, NJ (US)

(72) Inventor: Mark N. Hochman, Great Neck, NY (US)

(21) Appl. No.: 15/587,119

(22) Filed: May 4, 2017

(51) Int. Cl. A61M 5/158; A61N 1/36; A61N 1/05; A61B 5/00; A61M 5/145

(52) U.S. Cl. CPC A61M 5/158; A61N 1/36017; A61N 1/36021; A61N 1/0502; A61B 5/4848; A61M 5/1452; A61M 2205/3334; A61M 2205/581; A61M 2205/583; A61M 2205/582; A61M 2205/18; A61M 2205/502; A61M 2205/3344

(57) ABSTRACT

A system for infusing medication into a mammalian subject is provided. The system includes an injection system for controlling a flow of fluid from a fluid reservoir to a needle. A sensor is provided that detects a characteristic indicative of the fluid pressure in the needle. The injection system controls the flow of fluid to the needle in response to the characteristic detected by the sensor and the sensor continuously detects the characteristic as the needle is inserted into the subject. The system further includes a conductive element for providing electric nerve stimulation, wherein the system provides electric nerve stimulation in response to the sensor detecting a pressure exceeding an upper limit.

### FIELD OF THE INVENTION

The present invention relates generally to improvements to the delivery of drugs, particularly to systems for injection/aspiration into the body. More specifically, the invention provides a method and apparatus for determining the needle location when performing a Peripheral Nerve Block.

### BACKGROUND OF THE INVENTION

A peripheral nerve block (PNB) is used for anesthesia, postoperative analgesia, and diagnosis and treatment of chronic pain syndromes. Peripheral nerve blocks may also improve acute pain management and patient disposition even when used only as adjunct techniques. An objective of the PNB regional anesthesia technique is to identify the target nerve and position a hollow-bore needle in a defined proximity relative to the targeted nerve without causing untoward reactions such as structure damage to the nerve or causing excessive pain to the patient.

Referring to FIG. 1, a schematic description of the microanatomy of the peripheral nervous system is provided. The basic building block to both the central and peripheral nervous system is the single cell unit commonly known as is the axon. The brain and central nervous system are composed of millions of axons. Branching off the central nervous system of the brain stem and spinal cord is a collection of highly organized axons forming a network of sensory and motor pathways via the axons. At the emergence of the spinal canal this network of pathways is collectively known as the peripheral nervous system.

In the peripheral nervous system, each individual axon is surrounded by supporting connective tissue called the endoneurium. Contained within the endoneurium are small blood vessels (capillaries and venuoles) providing nutrients to these axons. Axons are collectively formed into highly organized, packed bundles that are surrounded by a thin but dense multi-layered connective tissue sheath that surrounds and forms a membrane structure called the perineurium. The perineurium provides a dense protective layer that is both a physical and chemical barrier, providing a degree of protection for the axons and endoneurium. This barrier is akin to the blood-brain barrier.

This discrete unit of the endoneurium and perineurium is called a peripheral nerve fascicle. When fascicles coalesce together they form fascicular bundles embedded in epineurium, which is a connective tissue sometimes referred to as inner epineurium. The multiple groups of fascicles are embedded in a non-uniform matrix of connective tissue (fibro-adipose tissue) and mid-size vessels that are loosely arranged together with an outer perimeter of dense connective tissue. The bundled fascicular structures collectively surrounded by this additional densely, more highly organized layer of fibrous tissue, houses the peripheral nerve contents and is known as the outer epineurium.

The outer epineurium connects the outer layer to the neighboring structures. A loose connective tissue fills the space between the nerve and the surrounding tissue in connection with the outer epineurium. There is thus an additional multi-layer boundary beyond the outer epineurium that runs along the entire trajectory of the nerve and is composed of an extraneural connective tissue known as the paraneurium. The paraneurium is a distinct multi-layer functional structure that enables the nerve to glide relative to other anatomic structures during muscular-skeletal movements.

To aid in locating a nerve branch, electrical stimulation may be utilized. Introducing an electrical current stimulation to the body has the ability to elicit an indirect excitation of both the sensory and motor components of a nerve. This was found to provide a visual muscle contraction when the electrical stimulation was applied and an electrical paresthesia when the electrical stimulation was applied on motor and sensory axons respectively. Modulating the charge frequency and current intensity lead to contraction and relaxation of muscle groups innervated by a nerve branch. However, this use of an indirect electrical charge to produce a nerve reaction to a specific nerve has not achieved widespread adoption because of several deficiencies, including:
An inability to accurately modulate an applied electrical charge at a given distances to the surface of a nerve branch has made nerve stimulation limited in the identification of a specific nerve branch when using nerve stimulation as the primary means of nerve branch location. A variety of charge intensities are recommended at specific distances when approaching the nerve branch. However, distance and intensity noted by a visual muscle twitch reaction does not correlate. Therefore, a reaction to a greater stimulation does not necessarily mean the needle is a greater distance to the intended nerve branch. And a reaction to a lower electric charge does not necessarily mean the needle is closer to the surface extraneural position and/or located within the nerve.
An inability to set the appropriate charge for a defined distance from the outer surface of the fascicle, i.e., Extra-Fascicular. It is more concerning if a high current charge is utilized near or Intra-Fascicular, as it may cause a forceful response by the patient. Hence there is an inability to determine what appropriate charge should be applied for a specific distance from the fascicle/nerve.
Confounding variables make the use of nerve stimulation a non-specific technique. These are related to anatomic variations within a given patient as well as anatomic variation between different patients. The body is comprised of a variety of tissue types which include connective tissue of mineralized and non-mineralized tissues. These tissues are composed of water and collagen, adipose tissue (fat), muscle, fluids (blood), bone, cartilage, etc. Each of these tissues types provides a different resistance and/or capacitance to a charge when it is applied at a given distance to the intended target.

In summary, the variables of charge intensity, frequency and tissue impedance to the electric charge have made it difficult to standardize a technique to enable precise needle to nerve distance and relationship with respect to the nerve layers of a specific nerve branch.

### SUMMARY OF THE INVENTION

In view of the shortcomings of the prior art, one aspect of the present invention provides a mechanism for distinguishing between intra-fascicular and extra-fascicular needle placement. Distinguishing between intra-fascicular and extra-fascicular can assist in avoiding an injection into a dense connective tissue close to or in the nerve, particularly the fascicles.

Another aspect of the current invention is a system in which continuous infusion of an ionic solution at a fixed flow-rate is provided as a fluid medium so that an electrical current charge can more effectively be transmitted thru these tissues. The ionic solution may be dispensed through a disposable syringe and tubing in a uniform way to the tip of the needle for the purpose of nerve stimulation conduction.

A further aspect of the current invention is a system that provides an ability to enhance ultra-sound visualization at the injection site by providing an increased fluid flow rate in response to increased fluid pressure as discussed further below. The increased fluid flow allows the tip of the needle to be more easily identified during the advancement of a needle through tissues when performing a peripheral nerve block, thus aiding in the prevention of needle tip placement into the nerve.

Another aspect of the current invention is controlling the electrical stimulation based on signals received regarding fluid pressure in the needle.

### BRIEF DESCRIPTION OF THE DRAWINGS

The foregoing summary and the following detailed description of the preferred embodiments of the present invention will be best understood when read in conjunction with the appended drawings, in which:

FIG. 1 is a cross-section view of a fascicle of nerve fibers;

FIG. 2 is a perspective view of a drug delivery system;

FIG. 3 is an enlarged side view of a handpiece of the drug delivery system illustrated in FIG. 2;

FIG. 4 is a diagrammatic view of an injection device of the drug delivery system illustrated in FIG. 2; and

FIG. 5 is a flow chart of a method for injecting fluid.

### DETAILED DESCRIPTION OF THE INVENTION

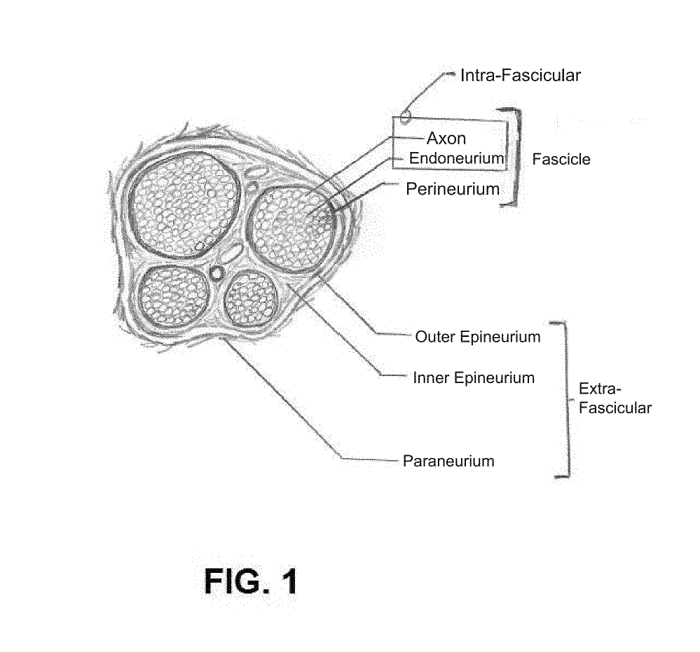

Referring now to the drawings, in general and to FIGS. 1-3 specifically, a drug infusion system is designated generally 10. The system 10 includes an injection assembly 20 and a computer-controlled drug delivery instrument 50, referred to as a drive unit. The injection assembly 20 includes an insertion needle 140 configured for insertion into a mammalian subject. The injection assembly 20 is connected with the drive unit 50, which controls the flow of fluid to the injection assembly during use. The system 10 also includes one or more output mechanisms that provide data to the medical professional during a procedure to assist in proper placement of the needle in the subject.

The system 10 is operable to determine the location for an intra-fascicular needle location. The system is also operable to deliver therapeutic medication to an extra-fascicular needle location. The medication may include, but is not limited to local anesthetic solutions, such as, cortico-steroids, hydroxyapatite, joint replenishment drugs, sclerosing agents and other drugs that are typically injected into a fluid-filled tissue space for therapeutic purposes.

An intra-fascicular needle location is one in which the tip of the needle penetrates through the perineurium so that the needle tip is located inside the fascicle. An extra-fascicular needle location is a position in which the needle is anywhere outside the perineurium of an individual fascicle, which may include outside the outer epineurium or even the paraneurium thereby defined as completely extraneural.

Irreversible damage can occur to a nerve when the needle tip is both embedded into a fascicle and then fluid under hydrostatic pressure produces changes to the neural and vascular tissues within the fascicle. This occurs because the outer layer of the fascicle is a protective layer of a relatively non-compliant, rigid protective structure. This protects the basic components of the nerve, the axons, which are densely packed within the fascicle. In other words, the fascicle represents a densely packed arrangement with a thick protective shell. The fascicle does not readily deform by either expanding or contracting. Therefore, tissue compliance to an inflow of fluids is extremely low and/or non-existent. Needle penetration into the fascicule may not necessarily cause the ultimate damage to the axon units, but the combined effect of needle penetration and increased pressure inside the fascicle from the infusion of fluids inside the fascicle can produce damage to the capillary bed. Additionally, fluid pressure-induced strangulation of the microcirculation of the axons impedes short-term nutrient replenishment after such physical trauma thus leading to initial necrosis. The cascade from necrosis leads to an inflammatory response in an effort to initiate a wound healing from the initial pressure-induced trauma further advancing or cascading potentially toward irreversible damage.

However, there are instances in which intentional intra-fascicular needle placement is desirable and required. Such instances include unresolved phantom pain after a limb is removed. Additionally, hyperactive neural stimulation of a particular limb may sometimes lead to retractable pain and is another circumstance in which intentional intra-fascicular needle location and delivery of agents is required. Accordingly, the system 10 and its use provide a method and apparatus for discriminating between the extra-fascicular and intra-fascicular location of a needle.

Injected fluid disperses through tissue at different rates. As a result, the fluid pressure varies. Therefore, this fluid pressure (or an internal pressure related to the resistance pressure of a tissue) is indicative of, and may be used to differentiate tissues of different densities.

The system 10 enables a practitioner to accurately identify fluid-filled tissue space while limiting the placement of drugs into non-targeted tissues. This is performed for both diagnostic and therapeutic procedures. The system 10 utilizes the pressure of a fluid as it flows from a needle or catheter following placement of the needle/catheter within the tissue in order to identify the accuracy of placement and to monitor the placement during an injection or aspiration. System 10 may utilize a continuous flow of fluid at what is considered a slow flow-rate that is defined as a constant flow-rate between 0.01 mL/sec to 0.20 mL/sec. The continuous flow of fluid maintains a constant column of fluid that may enable a virtually instantaneous reaction time to pressure changes within the tissues to be detected.

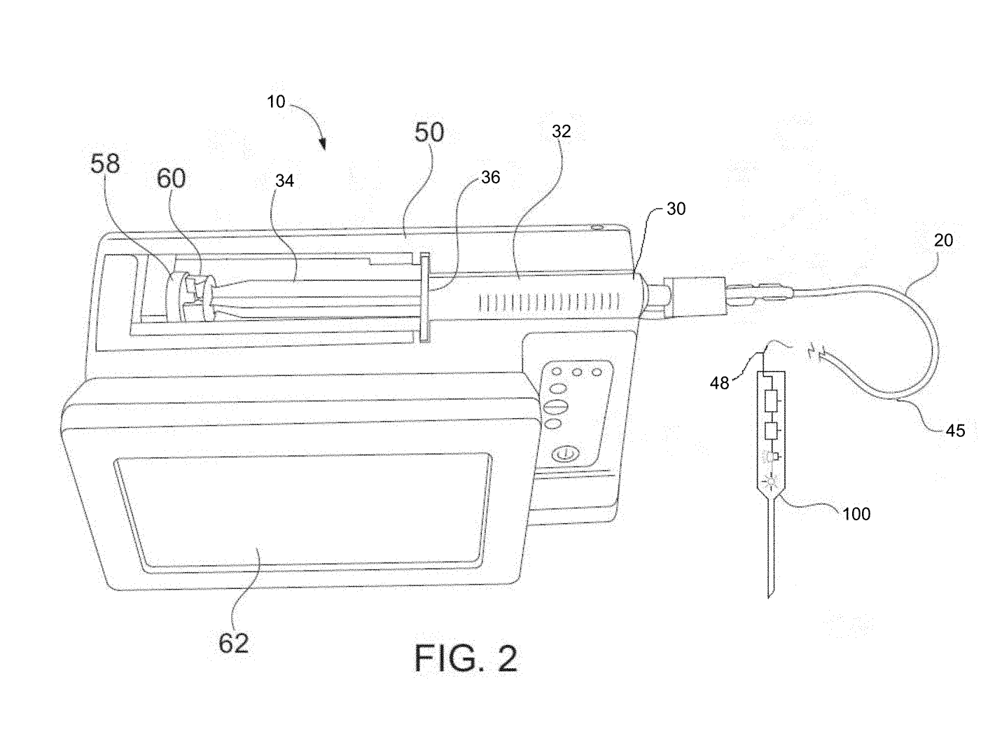

Specifically, the system 10 includes one or more output mechanisms for providing audible and/or visual feedback of the detected fluid pressure in the insertion needle. The operator uses the feedback as guidance during the placement of the insertion needle. As shown in FIG. 2, the first output mechanism may be a video display screen, such as an LCD display for displaying data to aid the operator. Additionally, a second output mechanism may also be provided. For example, the second output mechanism may be a speaker for providing an output signal.

### Injection Assembly

Referring to FIG. 2, the system 10 includes an injection assembly 20 cooperable with a drive unit 50 during a drug infusion procedure. The injection assembly includes a syringe 30, a handpiece 100, a fluid line 45 connecting the syringe with the handpiece and a cable 48 providing an electrical connection between the handpiece and the drive unit 50. The assembly further includes a needle 140 releasably connected with the handpiece 100.

Various elements of the injection assembly may be disposable, such as the syringe 30, the fluid line 45, the handpiece 100 and/or the needle 140. Alternatively, the elements may be re-useable. Accordingly, various elements of the injection assembly are releasably connectable. For instance, the fluid line 45 may include a fluid connector at each end. The fluid-tight connectors may be any of a variety of connectors. One exemplary connector is a Luer connector. At the first end, the fluid connector sealingly connects with the syringe and at the second end the fluid line sealingly connects with the handpiece 100. Alternatively, the fluid line 45 may be fixedly connected with the rearward end of the handpiece 100. In either embodiment, the handpiece 100 and the syringe are in fluid communication to provide a flow of fluid from the syringe to the handpiece.

The syringe 30 may be any of a variety of hypodermic syringes and the size may vary depending on the intended use. The syringe 30 includes a barrel 32 for holding a volume of medicament and a plunger 34 slidable within the barrel to draw fluid into or eject fluid from the barrel. The syringe 30 preferably also includes flanges 36 projecting outwardly from the barrel. The flanges operate as finger flanges to facilitate displacement of the plunger into the barrel.

The injection assembly 20 also includes a pressure sensor 40 for detecting fluid pressure in the injection assembly. The pressure sensor may be disposed in one of several locations to measure a pressure that correlates with the fluid pressure at the tip of the needle 140. Alternatively, rather than or in addition to an in-line pressure sensor, the pressure sensor may be a force sensor located within or connected to the thumb plate that drives the syringe plunger 58 or a force sensor that is internal to the drive unit 50 that measures the force applied to the syringe plunger. Such a force sensor detects the force required to inject the fluid, which is related to the fluid pressure the fluid pressure in the needle. Using such a sensor, the detected force is converted to a pressure value by a calculation via the processor. In the present instance, the pressure sensor 30 is an inline fluid pressure sensor attached to the syringe 30 between the syringe and the tubing 45. In this way, the pressure sensor 40 senses the fluid pressure as the fluid exits the syringe and enters the tubing 45 to which the insertion needle 140 is connected. Similarly, the in-line pressure sensor can be interposed between the tubing and the needle.

The injection system 10 may also include a re-useable handpiece 100 to which the needle 140 is attached. As shown in FIG. 2, the insertion needle 140 is connected to the forward end of the handpiece and the tubing 45 is connected to the rearward end of the handpiece. The handpiece 100 may be configured to provide electrical stimulation as discussed further below.

The injection assembly 20 may be manually operated to inject fluid. However, in the present instance, a computer-controlled drug delivery system 50 controls the flow of fluid from the injection assembly as discussed further below. An electrical cable 48 connects the pressure sensor 40 with the drug delivery system 50 so that the drug delivery system can monitor and, if desired, vary the flow of fluid from the syringe in response to the data from the pressure sensor 40. The pressure-transducer 40 may be connected inline between the forward end of the cylinder of syringe 30, and the first end of tubing 45. One exemplary connection is a Luer connection for connecting the pressure-transducer 40 to the tip of the syringe. The connection may be fixed by a threaded connection and/or an irreversible threaded connection, such as a LuerLok. Alternatively, the pressure transducer 40 may be permanently fixed to the syringe by plastic welding or chemical binding, such as adhesive. In this way, the instantaneous, actual fluid pressure in the drug delivery line 45 is sensed and used by the instrument, thereby providing a close approximation to the actual, instantaneous fluid pressure at the point or tip of the needle 140, and therefore, at the location in the patient's body where the needle tip is located. The electronic pressure-transducer 40 provides pressure data via an electronic data cable that is connected directly to the central unit 50 to collect the pressure measurements.

The electronic pressure transducer 40 can be any of various pressure sensors. One type of exemplary sensor is a piezoelectric pressure sensor, such as sensors available from Merit Medical Systems, Inc. such as the Meritrans® Pressure Transducer item MER212.

### Automated Fluid Delivery System

As described above, the system 10 may include a fluid delivery system 50 for providing a controlled flow of medication to the injection assembly 10. Preferably the fluid delivery system is an automated system and in the present instance is a computer controlled fluid delivery system referred to as a drive unit 50.

Referring to FIGS. 2 & 4, the drive unit 50 is designed to work in connection with an injection element, such as syringe 30. The drive unit 50 may include a cradle configured to receive the syringe 30 and a clamp for retaining the syringe in the cradle. The drive unit 50 includes a drive element 58 operable to drive the plunger in the syringe to expel fluid from the syringe. The drive unit 50 controls the displacement of the drive element 58 thereby controlling ejection of fluid from the syringe. In the present instance, the drive element may include a motor 70 driving an arm 58 having a clamp with a plurality of fingers that releasably engage the plunger 34. Driving the motor in a first direction drives the arm 58 forwardly to advance the plunger 34, thereby expelling fluid. The CPU 82 of the drive unit provides signals to the motor to control operation of the motor.

The drive unit 50 is operable to provide constant or variable fluid flow. In the present instance, the drive unit may provide a continuous fluid in response to signals received from the electronic pressure-transducer 40, which continuously senses the pressure of the fluid during an insertion/injection procedure. Based on a pre-determined pressure, the drive unit 50 may stop the flow of fluid when the detected pressure exceeds a pre-defined threshold. The pre-defined threshold may be set by the practitioner and stored in a memory 80 of a microprocessor or computer 82 of the electronics in drive unit 50. Similarly, based on a pre-determined pressure, fluid-flow will resume when the fluid pressure falls below a pre-determined pressure and will continue to flow while the pressure remains below the threshold. The same pre-determined pressure may be used to control the stopping and re-starting of the fluid flow. In such case, as fluid initially enters the tissue the pressure will build to a pre-determined level and then stop until once again the pressure drops below this pre-determined level. Once the fluid pressure falls below the pre-determined level, the fluid-flow will resume and be maintained on a continuous basis. In this way, the flow of fluid may start and stop during the procedure creating an interruption of fluid flow once a specific pre-determined pressure is detected

The system may include pre-defined pressure thresholds used to control the flow of medication from the syringe 30 during the procedure. This enables a clinician to selectively inject drugs into specific sites and intended tissues for diagnostic and therapeutic procedures. Pre-selected maximum allowable pressure limits and/or flow rates are stored in memory 80 and define either the maximum recommended pressures that patients usually tolerate, or other criteria. As the pressure approaches this limit, a visual and/or audible alarm is generated for the clinician, i.e. on screen 62 and via speaker 84 that is activated by data from the microprocessor 82. In addition, data descriptive of the whole injection process is stored for future analysis in memory 80.

The system 10 may directly measure the fluid pressure in the injection assembly 10 or the system may measure a characteristic indicative of the fluid pressure in the injection assembly. For instance, the pressure may be measured by detecting the pressure resistance measured during infusion. The pressure resistance measured is converted into a visual signal on a continuous basis during the insertion procedure. The flow rate of medication during the procedure may be based on the fluid pressure detected in real time during the procedure. Therefore, the flow rate of the medication may be variable and may be dependent on the pressure in the system. In this way, the fluid pressure may be the primary controlling variable of the system.

One feature of the present system is the ability to detect minute changes in pressure at the needle tip while a needle is placed within the patient's tissue. This ability to detect subtle pressure changes is based upon a constant movement of fluid into the tissues under controlled conditions thus enabling one to identify and or avoid undesirable locations based on pressure within the tissue. The system detects these minute changes in pressure in real-time and dynamically when a continuous flow of the fluid is used. This continuous flow is coordinated with a pre-determined maximum pressure used by the system to stop the flow of fluid at a pre-determined pressure limit to avoid damage to these tissues. With a constant flow of fluid the head pressure provides the needed resistance within the tissues to enable subtle changes in the tissue density and compliance to be detected on a virtually instantaneous basis.

The flow-rate, therefore, becomes a second variable that is modulated within a pre-determined range in order to maintain the desired fluid-flow. In one specific embodiment, the fluid flow is stopped when the pressure exceeds a pre-determined threshold (maximum pressure). The flow-rate, as a second variable, may be limited so that fluid injections are not unduly rapid under low pressure conditions. It is contemplated that the relationship between pressure and fluid flow rate may either be binary or continuous. A binary relationship exists when the injection device is configured to deliver fluid at a single, pre-determined flow rate for any pressure less than the pre-set maximum. Thus, the fluid flow is either on or off based on whether or not the pressure exceeds the threshold. Alternatively, the flow rate may be modulated as a function of pressure. In this case, flow rate will be reduced as the maximum pressure is approached and increased as the pressure drops. Optionally, the flow rate may be limited to a first pre-set maximum pressure and a flow rate resumes at a second distinct pre-determined pressure.

As mentioned above, the system 10 may include a mechanism for displaying relevant injection data including, for example, instantaneous flow rates, pressures, and injection amounts upon a screen 62 of the drive unit 50. Similarly, the system may include a mechanism for recording such information for subsequent analysis after the procedure is performed. For instance, the system may include a non-volatile electronic storage medium, such as a hard drive, flash drive, optical drive or other medium for storing electronic data.

All measurements and information may be presented to the clinician in “real-time” so that the clinician may determine whether the injection is being delivered to the intended location and/or correct tissues and may modify the injection technique accordingly. In addition, the measurements may be recorded for later review and documentation of the clinical event.

It is also contemplated that multiple syringes driven by separate syringe plungers may be used to allow multiple drugs to be injected as well as a second syringe drive that does not required a pre-determined pressure to be reached for any said purpose. The second drive can be programmed on a specific flow-rate to allow infusion of a drug such as local anesthetic and other therapeutic drugs into a variety of tissues.

In yet another embodiment the device may contain two distinct syringe drives in which both are capable of modulation based on fluid-pressure as previously herein described.

### Electrical Stimulation

The system may also include an electrical stimulation element for providing electrical nerve stimuli to a target tissue in a patient. The electrical stimulation element is a conductive element connected with the handpiece 100. The electrical stimulation element is operable to provide an electrical charge of low intensity (i.e. approximately 0.10 mA up to approximately 2.0 mA) and short duration (i.e. pulses of approximately 0.05 to 1 Ms) and low frequency 1 or 2 Hertz. The electronic stimulation elements provide the stimuli for a short time (i.e. approximately 1-10 seconds).

The electric stimulator may be an external element or an internal element. For example, FIGS. 2-4 illustrate an embodiment that incorporates external electric stimuli. A conductive element such as an electrically conductive cable 48 interconnects the handpiece 100 with a stimuli generator 85, so that electrical stimuli are transmitted to the handpiece from the stimuli generator. In turn, the handpiece is connected with an element configured to deliver the electrical charge to the tissue. For instance, the needle 140 may be formed of electrically conductive material and the handpiece may include a connection with the needle providing an electrical pathway from the conductive element 48 to the needle. Alternatively, a conductive element, such as a wire, may extend along the length of the needle and the needle may be electrically insulated from the conductive element. For example, the needle may be coated and the coating be formed of electrically insulative material. The outside shaft of the needle is coated with an insulated coating and the needle tip and the inside of the needle are not coated. An example of an external electric stimulation element is the insulated needle sold under the trade name “Stimuplex®” or the over the needle catheter sold under the trade name “Contiplex® C” by B. Braun Medical Inc. of Bethlehem, Pa.

The system may utilize internal electric stimuli rather than the external electric stimuli described above. For example, the fluid injected from the syringe may be an ionic solution capable of conducting electric stimuli. A conductive element may be interconnected with the fluid within an insulated needle. The needle may be constructed from a variety of non-conductive materials. For instance, the conductive element may project into the fluid path at some point between the syringe 30 and the needle 140. For example, the conductive element may impart the electric stimuli into the fluid at the rearward end of the handpiece 100. If the electric stimuli are imparted to the tissue via the fluid, the needle 140 may be electrically insulated to minimize any drain or disbursement of the electric charge through the sidewalls of the needle.

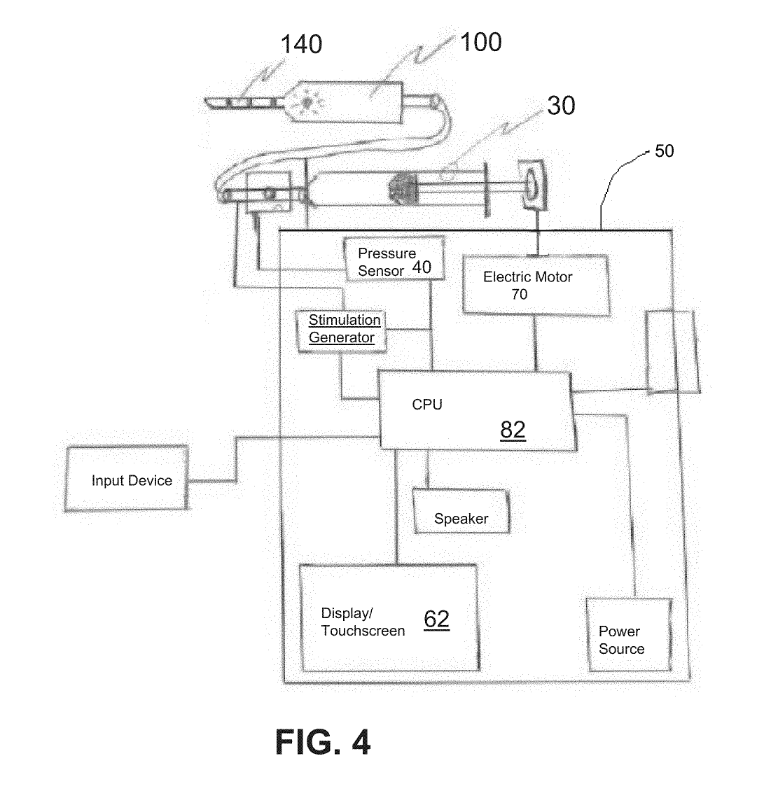

As shown in FIGS. 2 & 4, the electrical stimulation element is connected with an electric stimuli generator 85, which is an electrical source operable to provide an electrical shock or pulse to the stimulation element. The stimuli generator may be incorporated into the drive unit 50 as shown in FIG. 4. In such an arrangement the stimuli generator 85 is connected with the CPU of the drive unit so that the CPU provides electric signals to control the operation of the stimuli generator. Alternatively, the stimuli generator may be a separate element having a separate power source and separate control.

### Calculation of Fluid Pressure at the Exit of the Needle

As discussed above, the fluid pressure may be used to control operation of the system 10. For instance, the system may provide a signal to the operator when the fluid pressure exceeds a threshold, thereby indicating that the needle may be located intra-fascicularly or the needle may be indenting or up against the epineurium. There are several methodologies for calculating the fluid pressure at the exit of the needle.

A pressure sensor may detect the fluid pressure in the injection assembly 100. For example, as discussed above the pressure sensor may be an in-line pressure sensor, such as that available by Merit Medical part #0001. Alternatively, a pressure sensor internal to the drive unit 50 may detect the fluid pressure between the syringe 30 and the tubing 45. Similarly, the pressure sensor can be interposed between the syringe tubing 45 and the needle 140. Further still, the in-line sensor may be embedded into the handle 100 or between the tubing 45 and the handle 100. Another alternative is using a thumb-pad force sensor to detect the force driving the plunger 34 to calculate the pressure within the syringe 30. A command signal from the pressure sensor sends data of pressure to the CPU for calculation to determine the exit-pressure. The exit-pressure value is used to control the motor 70 that controls the flow of fluid from the syringe 30.

### Handpiece

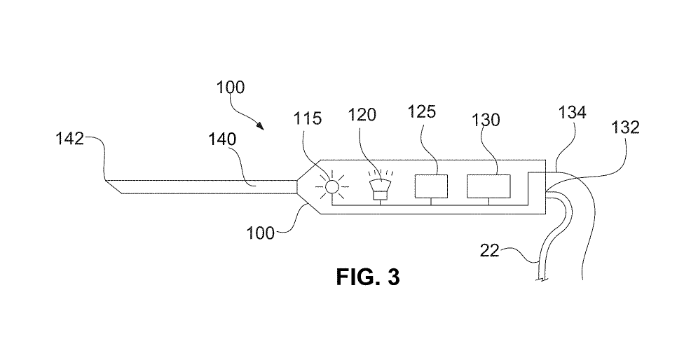

Referring to FIG. 3, the handpiece 100 includes a hollow housing 110 and an elongated hollow needle 140 projecting forwardly from the housing. A connector 132 is provided for connecting the handpiece with the fluid line 45 of the injection assembly 10. Specifically, the connector 132 provides a fluid-tight seal for connecting the handpiece 100 at the rearward end of the housing to facilitate connection of the handpiece with the fluid in the syringe. The fluid flows to the handpiece and out through the needle 140. As noted above, the in-line pressure sensor or similar element for detecting a characteristic representative of fluid pressure may be embedded within the handpiece 100.

The handpiece 100 may further include an indicator light 215 configured to provide the operator with prompts. The indicator light 115 may be an LED or other light element that provides a warning light or indicator light depending upon the application. The handpiece may further include an audible indicator 120 such as a piezoelectric audio indicator for providing an audible signal, including, but not limited to a buzz, tone or chime.

Additionally, a control button 125 may be provided for the handpiece. The control button 125 may operate as an on/off button. However, the control button may also be operable to enter various control commands. For instance, the control button 125 may be operable to over-ride one or more operations of the drive unit 50 as discussed further below. Finally, the handpiece 100 may also include an output mechanism, such as a display screen 130 for displaying various information, such as the detected fluid pressure.

As described above, the handpiece 100 may include both a visual and an audible indicator 115, 120. It should be understood that the handpiece does not need to include both an audible and a visual indicator; it could include just a single indicator. Further still, although a visual and audible indicator are described, a variety of alternate indicators could be used instead, such as a vibration element that provides regular vibration indicator signals. Additionally, the handpiece 100 need not include any such indicators.

As noted above, the handpiece 100 may include a control button. The control button may be utilized when the needle is not being advanced. In such an instance, pressing the button operates to provide a control signal to the drive unit 50 so that a counter-head pressure value will not be subtracted from the calculation of the exit-pressure (since the needle is not being advanced there is zero, or essentially zero, counter-head pressure). It is understood that the button or control on the handpiece 100 may also be activated to correspond with the forward movements in which the counter head-pressure is subtracted from the calculation of the head-pressure therefore providing a means to distinguish between when the needle is being advanced and when it is remaining stationary within the tissues. In this way, actuation of the button 125 during periods of minimal to zero needle insertion promotes accuracy of the exit-pressure values within the tissues during the procedure. In addition to the switch or control button discussed above, the handpiece may include a second button or control element in which backward movements would add an additional head-pressure value to compensate for the backward movement which causes a decrease in exit-pressure values when moving a needle backward through the tissues.

In the foregoing description, the needle is mounted onto a handpiece that may have additional features. However, it should be understood that various elements may be utilized to carry the needle. For instance, the system may be utilized with an embodiment in which the needle is connected to the tubing set. In such embodiments, the operator can control the intensity of the electrical charge by a controller that is operable independently of the handpiece, such as by a controller on the control unit 50.

### System Control

The system includes a user operable input mechanism, which allows the operator to provide input signals for controlling the system. The input mechanism may be any of a variety of devices, such as the handpiece 100 or a foot operated control that provides a means for the operator to start, stop, and change the flow-rate from a single flow-rate to a second or third distinct pre-set flow rate. Alternatively, the input element may be a button, touchscreen, mouse, keyboard or a microphone for providing input commands audibly. Additionally, the system may include a plurality of input mechanisms to allow the operator to input a variety of inputs for various stages of a procedure. For example, the system may include a first input mechanism, such as a foot pedal that controls the flow of fluid through the device. Actuating the foot pedal switch (i.e. depressing the switch) sends a signal to the CPU of the drive unit, which in turn sends a signal to the motor to drive the motor so that fluid flows from the syringe to the needle 140 as long as the pedal is actuated. Alternatively, actuating the foot pedal a first time may operate a start signal to start the fluid flow and the fluid may continue to flow until the operator actuates the foot pedal again. In this way, the second actuation operates as a stop signal to discontinue the fluid flow. Additionally, the system may include a second input mechanism, such as a touch screen so that once an electronic simulation is applied to a patient the operator may input an indication of whether or not muscle twitch was detected or whether a sensation is noticed by the patient. Actuating the foot pedal switch (i.e. depressing the switch) may send a signal to the CPU of the drive unit, which in turn sends a signal to the controller of the electronic stimulator to vary the intensity of the charge. These operator control signals can work separately from one another, i.e., effecting either fluid flow or intensity of stimulation. Further still, the primary or secondary input mechanism may be a control button, such as button 125 on the handpiece. Actuating the control button 125 may send a signal to the CPU to provide a response input during a procedure.

As described above, the system is operable to control the flow of fluid during a procedure. In addition to using an actuator to control On/Off, the system may provide two or more flow rate settings. In particular, the control unit 50 may incorporate a multi-speed pump that provides a variable flow rate. Similarly, the pump may include two or more pre-set flow rates. In the present instance, the control unit 50 includes an electric motor 70 that control the speed at which the control unit displaces the plunger 34 in the syringe 30. The control unit 50 may control the speed of the motor 70 so that the motor is driven at one of multiple pre-set speeds to provide multiple pre-set flow rates. The different flow rates can be used in conjunction with different pressure settings and different electric stimulation settings during different portions of a procedure. For example, the system may be configured with three pre-sets as shown below in the table. The pre-sets may include various characteristics, such as high and low pressure thresholds, high and low stimulation intensities, flow rate and maximum or shut-off pressure. When the measured fluid pressure is above the “Low Pressure” threshold, electric stimulation is provided at the level identified as “Low Pressure Stimulation”. When the measured fluid pressure is above the “High Pressure Threshold, electric stimulation is provided at the level identified as “High Pressure Stimulation”. The “Flow Rate” is the flow rate of fluid provided from the fluid reservoir (e.g. syringe 30). The “Shut-off Pressure” is the fluid pressure at which the system will interrupt fluid flow. Specifically, if the measure fluid pressure exceeds the shut-off pressure, the control assembly 50 will stop the pump to stop the flow of fluid to the needle.

| | | | Low | High | | |
|---|---|---|---|---|---|---|
| | Low | High | Pressure | Pressure | | Shut-off |
| | Pressure | Pressure | Stimulation | Stimulation | Flow Rate | Pressure |
| | (mm/Hg) | (mm/Hg) | (amperes) | (amperes) | (mL/sec) | (mm/Hg) |
| Pre-set 1 | 50 | 200 | 0.4 | 1.0 | 0.02 | 300 |
| Pre-set 2 | 75 | 400 | 0.7 | 1.4 | 0.1 | 500 |
| Pre-set 3 | 100 | 650 | 1.0 | 1.8 | 0.2 | 750 |

As noted above, the different pre-sets may be used during different portions of a procedure. For example, this first pre-set may be used during a first portion of a procedure, such as the portion of the procedure in which the operator is attempting to position the tip of the needle adjacent the target nerve. During this “Locate the Target” portion of the procedure a constant low “Flow Rate” is used to allow for increased sensitivity to the pressure changes. After the needle is positioned adjacent the intended target, the system may switch to a second pre-set. During the second pre-set, the Flow Rate is increased to provide a confirmation of the needle placement. In particular, if the system is used in connection with ultrasound, the location of the needle may be difficult to detect depending on a number of variables, such as the orientation of the needle. However, if a rapid infusion of fluid is provided as defined in pre-set 2, the fluid will appear on the ultrasound display as a dark anechoic pocket of fluid. In this way, the location of the pocket of fluid on the ultrasound image will indicate the location of the needle. Additionally, the particular shape of the fluid on the ultrasound image may provide confirmation that the needle is extra neural rather than intraneural. For instance, a donut-shaped fluid space can confirm that the needle is in close proximity to the nerve and extraneural. Therefore, seeing such a shape may provide confirmation of proper needle placement. It should be understood however, that a variety of shapes can be used as confirmation that the needle is extraneural.

After the needle position is confirmed using the second or “Confirmation” pre-set, the third pre-set (Pre-set 3), referred to as the “Infusion” pre-set is used. This third pre-set has a higher Flow Rate so that the bolus of medication can be rapidly infused at the target location adjacent the nerve for maximum efficiency.

The switch between pre-sets can be manual or automatic. For example, the operator may manipulate an input device, such as a keyboard, touch pad, a button on the handpiece or otherwise, as noted above. Alternatively, the system may automatically switch to the second pre-set based on detected criteria, such as the fluid pressure, electrical impedance, or change in electrical impedance.

The control unit 50 may be preconfigured with different pre-set characteristics, such as the pre-sets described above. For example, the pre-set characteristics can be set by the operator before a procedure or the pre-set values may be pre-programmed into the system. Additionally, the system may allow the user to modify the pre-set characteristics during use. Further still, it should be understood that any number of pre-sets may be utilized. Three pre-sets are described above; however, the system may use fewer pre-sets. For instance, the system may not include any pre-sets and the user may simply change the different values during a procedure. Alternatively, the system may be programmed to include only two pre-sets, such as the “Target Location” pre-set and the “Infusion” pre-set. Further still, the system may include four or more pre-sets that include different operating characteristics for different applications.

### Method of Operation

An exemplary method for administering a nerve block injection using the system described above will now be described. It should be understood that the present system is not limited to use in peripheral nerve block procedures. Accordingly, it should be understood that the principles and methods described below may be readily adapted for injections into tissues and anatomical areas in a variety of applications and procedures.

The system may be used to detect whether the needle is positioned within the fascicle (i.e. positioned intra-fascicularly). The system makes the determination based on a combination of several variables. First, if the needle has pierced the endoneurium the fluid pressure will be quite high because the axons are tightly packed within the endoneurium and the perineurium defines a boundary of a rigid non-compliant component. Additionally, if the needle has pierced the endoneurium the operator is likely to observe a noticeable response to an electrical stimulation applied to the patient at or adjacent the needle tip. Further still, as the needle approaches the nerve, the nerve may respond to a lower intensity electrical charge. Therefore, various features may be monitored to determine whether the needle is positioned intra-fascicularly and therefore should be re-positioned. Therefore, the system may operate as follows.

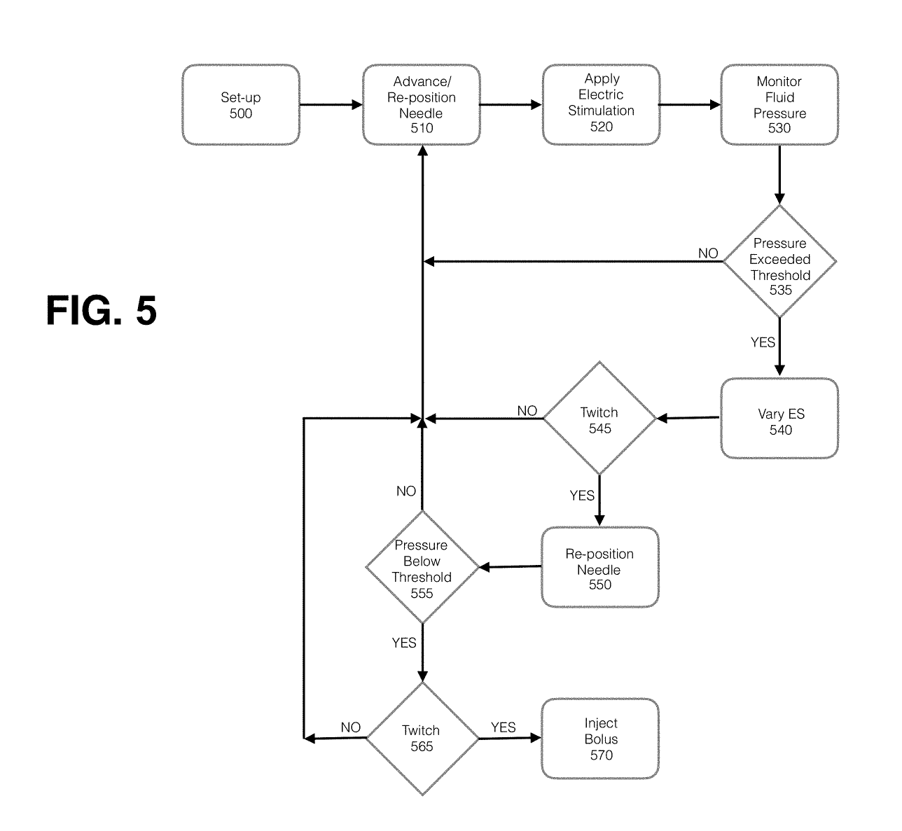

Referring to FIG. 5, at step 500 the operator selects the procedural parameters, such as the upper threshold and/or the fluid flow rate and/or the fluid pressure at which electrical stimulation commences. For example, the operator may set an upper threshold pressure, such as 300 mm/Hg and the operator may set a threshold for commencing electrical stimulation at 50 mm/Hg. Alternatively, the upper threshold may be pre-set in the system when the operator selects the type of procedure for which the system is to be used. Similarly, the operator may select the fluid flow rate through the needle or the flow rate may be set automatically when the operator selects the type of procedure. Additionally, the operator may select characteristics of the electrical nerve stimulation that is to be applied, such as frequency, high intensity, low intensity, etc. Once the procedural parameters are selected, the operator provides an indication that the procedure is to start. For instance, the operator may press a start button on the drive unit.

At step 510 the operator advances the needle into the patient. The needle may be advanced at any of a variety of insertion rates, such as 1-3 mm/sec. Preferably, the needle is inserted at a substantially constant rate. Additionally, the control system provides a continuous flow of ionic fluid through the needle tip during the procedure. The flow rate may vary depending on different parameters as discussed previously. For instance, the fluid flow may be infused at approximately 0.02 mL/sec unless the fluid pressure in the needle exceeds a maximum threshold.

At step 520 electric stimulation (ES) is applied. For instance, the electrical stimulation is applied via the needle tip of an insulated needle. The ionic fluid infused through the needle reduces the impedance, which can reduce pain or discomfort that the patient may feel from the electric stimulation. The intensity and frequency of the electric stimulation may vary depending on the parameters established during set-up step 500. Although FIG. 5 shows the electric stimulation as starting once the needle is inserted into the patient, it should be understood that the electric stimulation may not commence until later in the procedure. For instance, the electric stimulation may not commence until the measured fluid pressure exceeds a threshold. In such a method, the electric stimulation would not commence until step 540 as discussed below.

At step 530 as the operator advances the needle, the system continuously determines the fluid pressure at the needle tip and provides feedback either visually or audibly regarding the determined pressure. For example, as described above, the system may include a pressure sensor 40 operable to measure the fluid pressure inline with the needle. The sensor may provide signals to the control unit 50 that may be used to control the electric stimulation.

At step 535 the system compares the detected fluid pressure with a pre-determined threshold. In the present instance, step 535 occurs during infusion of the drug. If the detected fluid pressure does not exceed the threshold then the method returns to step 510 and the operator continues to advance the needle to attempt to locate the target nerve. If the system detects that the fluid pressure exceeds the threshold then the method proceeds to step 540.

At step 540 the pressure exceeds a threshold so a characteristic of the electrical nerve stimulation is varied. For instance, as noted above, the electrical stimulation may not commence until the fluid pressure exceeds a first threshold. If the fluid pressure exceeds the first threshold then the system commences electrical stimulation at step 540. Similarly, if the fluid pressure exceeds a second threshold then the system varies the intensity of the electrical stimulation at step 540 and continues to apply electrical stimulation. For instance, the system may be configured so that the electrical stimulation is applied at 1.0 mA when the electrical stimulation commences. If the fluid pressure exceeds a second threshold then the intensity of the electrical stimulation may be reduced, such as by lowering the electrical stimulation to 0.4 mA.

At step 540 the electric nerve stimulation may be varied manually or automatically by the system in response to the fluid pressure exceeding the upper threshold. For instance, once the fluid pressure exceeds the second threshold the system may automatically vary the electrical stimulation (such as by commencing or by varying the intensity). Alternatively, the system may provide a signal to the user (such as an audible or visual signal) and the operator may vary the electric stimuli by manipulating a control element such as a button or touchscreen. In response to the operator's prompt, the electric nerve stimulation is varied and applied to the patient.

At step 545 the operator monitors the patient to detect any clinically observable response, such as a muscle twitch. If the operator does not observe a twitch in response to the electric stimulation then the method returns to step 510 and the operator continues to advance the needle. If the operator observes a noticeable twitch then the needle may be adjacent the target nerve. However, since the fluid pressure exceeds the second threshold, the needle may be intrafascicular, which could lead to damage or complications if the anesthetic is injected while the needle is in the nerve. Accordingly, if the operator observes a twitch at step 545 the method may proceed to step 550.

At step 550 the operator repositions the needle in an attempt to position the needle tip adjacent the nerve but not in the nerve or in direct contact with the surface of the perineurium of the nerve. For instance, the operator may withdraw the needle slightly and then attempt to position the needle tip adjacent the nerve. The step of repositioning may be guided by ultrasound so that the operator may be able to visually confirm that the needle is withdrawn from the nerve and then repositioned adjacent the nerve. If the needle was positioned within the nerve, the fluid pressure would exceed the second threshold as described above. As in example Pre-set 1, if the pressure exceeds the pressure threshold value 300 mm/Hg and the operator detected a muscle twitch from a reduction in the electrical stimulation intensity then the system will provide a warning or alarm and prevent further infusion of fluid by the operator. If the needle is then withdrawn then the fluid pressure should fall below the threshold so fluid flow can re-commence. Accordingly, after re-positioning the needle the method moves on to step 555.

At step 555 the fluid pressure is evaluated to determine whether the fluid pressure exceeds the second threshold. If the fluid pressure exceeds the second threshold, as by this example 300 mm/Hg (Pre-set 1), then the needle tip may still be within the nerve. Accordingly, the method returns to step 550 so that the needle may be re-positioned. Alternatively, the needle may be withdrawn and the process may re-start at step 510. If the step of re-positioning the needle has withdrawn the needle tip from being within the nerve (i.e. intrafascicular), then the withdrawal of the needle will cause the fluid pressure to drop below the second threshold. Therefore, if the fluid pressure drops below the second threshold then the method proceeds to step 565.

At step 565 the electrical stimulation is applied and the operator observes whether a twitch is observed in response to the electrical stimulation. If the operator observes a twitch, the operator may then provide an input to the system indicative of whether an observable response was detected or not. For instance, the operator may press a first button if the operator noticed a twitch or the operator may press a second button if the operator did not notice a twitch. It should be noted that the electrical stimulation that is applied is the electrical stimulation that was varied at step 540. For example, if the fluid pressure exceeded the second threshold and the intensity of the electrical stimulation was decreased, then the electrical stimulation will continue to be applied through step 565. Therefore, if the operator observes a twitch at step 565 the operator has confirmed that the nerve has responded to the varied (i.e. increased or decreased intensity) electrical stimulation and that the fluid pressure is consistent with being extraneural. Accordingly, if the operator observes a twitch in response to the electrical stimulation then the method proceeds to step 570. If the operator does not observe a twitch then the procedure returns to step 510 to re-start the search for the target nerve.

At step 570 the system provides a signal to the operator indicating that the needle is properly located for an injection (i.e. the needle tip is located extra-fascicularly). For example, the control unit 50 may provide an audible signal such as announcing the word “proceed” or providing a visual signal, such as the word “proceed” on the display screen of the drive unit or the handpiece.

At step 570 the flow rate of fluid is increased to a second rate that is higher than the first rate. The operator may inject a preliminary amount that may be observable so that the operator may detect that the needle is properly placed. Once placement is verified, the operator may inject a bolus of fluid to anesthetize the patient. Alternatively, the operator may inject the bolus of fluid without first injecting an amount to verify the needle placement. Either way, a quantity of fluid is injected at step 570 at a higher rate than the previous low flow rate. For instance, the drive unit may automatically increase the flow rate, such as by increasing the speed of the motor

As discussed above, the system may combine various measured characteristics to assess whether the needle is positioned adjacent the target nerve and whether the needle is intrafascicular or extrafascicular. In the exemplary method illustrated in FIG. 5, the method incorporates features regarding the fluid pressure at the needle tip as well as the intensity of the applied electrical stimulation.

It should be understood that the values provided above, such as the threshold of 300 mm/Hg as the maximum pre-set pressure for stoppage of fluid flow is an example and that either a lower or higher pre-set pressure may be selected at the discretion of the clinician. Similarly, the values of 0.4 mA and 1.0 mA for the lower and upper electrical intensities are examples and either higher or lower values may be set. However, in the present instance, it may be desirable to select an upper amperage that does not exceed 2.0 mA. The techniques described herein are equally applicable to human and animal tissues.

It will be recognized by those skilled in the art that changes or modifications may be made to the above-described embodiments without departing from the broad inventive concepts of the invention. For instance, in the foregoing description, the system is described in the context of providing fluid infusion. However, it should be understood that the system may be used for placement of a needle to aspirate fluid-filled tissue. It should therefore be understood that this invention is not limited to the particular embodiments described herein, but is intended to include all changes and modifications that are within the scope and spirit of the invention as set forth in the claims.

What is claimed is:

1. An apparatus for injecting medicinal fluid, comprising:
an injection system for controlling a flow of fluid from a fluid reservoir to a needle, wherein the needle is configured for subcutaneous insertion into a mammalian subject;
a sensor for detecting a characteristic indicative of the fluid pressure in the needle, wherein the sensor is configured to continuously detect the characteristic as the needle is inserted into the subject; and
an electric nerve stimulation element for providing an electric nerve stimulation at or adjacent to a tip of the needle;
wherein the electric nerve stimulation element varies the intensity of the electric nerve stimulation to a patient after the sensor detects a characteristic of the fluid pressure indicative of the fluid pressure exceeding a first threshold.

2. The apparatus of claim 1 wherein the system includes an indicator operable to provide an audible, visual or tactile signal.

3. The apparatus of claim 2 wherein the indicator is operable to provide a signal when the fluid pressure exceeds the first threshold.

4. The apparatus of claim 2 comprising an input element configured to provide a mechanism for an operator to input whether a clinically observable response was observed in response to the electric nerve stimulation after the intensity is varied.

5. The apparatus of claim 4 wherein the indicator is operable to provide a signal indicative of the needle being in an appropriate position for an injection, wherein the indicator is configured to provide the signal in response to a signal from the input element indicative of no clinically observable response being observed.

6. The apparatus of claim 1 wherein the injection system includes a fluid reservoir and an elongated flexible tube, wherein a first end of the flexible tube is connected with the fluid reservoir and a second end of the flexible tube is connected with the needle.

7. The apparatus of claim 6 wherein the sensor is located in-line between the fluid reservoir and the needle so that the sensor detects the fluid pressure in-line with the flow of fluid between the reservoir and the needle.

8. The apparatus of claim 1 wherein the injection system comprises a microprocessor for controlling the rate of fluid flowing from the fluid reservoir.

9. The apparatus of claim 1 wherein the electric nerve stimulation element is configured to reduce the intensity of the electric nerve stimulation to a patient after the sensor detects a characteristic of the fluid pressure indicative of the fluid pressure exceeding the first threshold.

10. The apparatus of claim 9 wherein the electric nerve stimulation element is configured to reduce the intensity of the electric nerve stimulation from no greater than approximately 2.0 amperes to no greater than approximately 1.0 amperes after the sensor detects a characteristic of the fluid pressure indicative of the fluid pressure exceeding the first threshold.

11. The apparatus of claim 9 wherein the electric nerve stimulation element is configured so that the electric nerve stimulation element does not provide electric nerve stimulation unless the sensor detects a characteristic of the fluid pressure indicative of the fluid pressure exceeding a second threshold that is lower than the first threshold.

12. The apparatus of claim 11 wherein the second threshold is less than or equal to approximately 100 mm/Hg.

13. A method for providing a peripheral nerve block to a patient, comprising the steps of:
inserting a needle into a patient;
providing a flow of fluid through the needle while the needle is in the patient;
monitoring the fluid pressure in the needle while the needle is in the patient;
providing an electric nerve stimulation at or adjacent to a tip of the needle in response to the signal; and
varying the intensity of the electric nerve stimulation in response to the fluid pressure exceeding an upper limit.

14. The method of claim 13 comprising the step of monitoring the patient to detect a response to the electric nerve stimulation after the step of varying the intensity.

15. The method of claim 14 comprising the step of providing a first signal if a response to the electric nerve stimulation is detected or providing a second signal if no response to the electric nerve stimulation is detected.

16. The method of claim 15 comprising the step of re-positioning the needle in response to the first signal.

17. The method of claim 14 wherein the step of varying comprises reducing the intensity of the electric nerve stimulation.

18. The method of claim 17 wherein the step of varying comprises reducing the intensity of the electric nerve stimulation from no greater than approximately 2.0 amperes to no greater than approximately 1.0 amperes.

19. The method of claim 13 wherein the step of providing electric nerve stimulation does not occur until the step of monitoring the fluid pressure indicates that the fluid pressure exceeds a lower threshold that is lower than the upper threshold.

20. The method of claim 19 wherein the lower threshold is less than or equal to approximately 100 mm/Hg.

21. A system for providing a peripheral nerve block to a patient, comprising:
a needle having a sharpened tip;
a fluid pump providing a flow of fluid to the needle,
a controller for controlling the fluid pump to control the flow of fluid to the needle;
a sensor for detecting the fluid pressure in the needle;
a conductive element for providing an electric nerve stimulation at the tip of the needle; and
an output element configured to provide a human perceptible signal;
wherein the controller is configured to vary the intensity of the electrical nerve stimulation after the sensor detects a fluid pressure exceeding the upper limit.

22. The system of claim 21 comprising an input element configured to allow the operator to indicate whether a clinically observable response was observed in response to the electric nerve stimulation.

23. The system of claim 22 wherein the controller is configured to control the output element to provide a warning signal in response to operator input to the input element after the controller varied the intensity in response to the sensor detecting a fluid pressure exceeding the upper limit.

24. The system of claim 21 wherein the fluid reservoir comprises a syringe having a plunger and the injection system comprises a control mechanism for automatically advancing the plunger to expel fluid from the syringe.

25. The system of claim 21 wherein the sensor comprises a pressure transducer.

26. The system of claim 25 wherein the sensor detects fluid pressure and the pump controls the flow of fluid in response to the detected fluid pressure.

27. The apparatus of claim 21 wherein the electric nerve stimulation element is configured to reduce the intensity of the electric nerve stimulation from no greater than approximately 2.0 amperes to no greater than approximately 1.0 amperes after the sensor detects a characteristic of the fluid pressure indicative of the fluid pressure exceeding the upper threshold.

28. The apparatus of claim 21 wherein the electric nerve stimulation element is configured so that the electric nerve stimulation element does not provide electric nerve stimulation unless the sensor detects a characteristic of the fluid pressure indicative of the fluid pressure exceeding a lower threshold that is lower than the upper threshold.

29. The apparatus of claim 28 wherein the lower threshold is less than or equal to approximately 100 mm/Hg.
